(19) United States
(12) Patent Application Publication
(10) Pub. No.: US 2025/0242169 A1
(43) Pub. Date: Jul. 31, 2025

(54) Micro LED Skin Treatment Built-in Lip Balm

(71) Applicant: L'Oreal, Paris (FR)

(72) Inventors: Gregoire CHARRAUD, Jersey City, NJ (US); Tara Ferrero, Sceaux (FR); Paul Tartrat, Paris (FR)

(73) Assignee: L'Oreal, Paris (FR)

(21) Appl. No.: 18/428,849

(22) Filed: Jan. 31, 2024

(51) Int. Cl. A61N 5/06

(52) U.S. Cl. CPC A61N 5/0616; A61N 2005/063; A61N 2005/0644; A61N 2005/0652

(57) ABSTRACT

Devices and methods for skin light therapy via light emitting diodes (LEDs) in combination with application of a cosmetic to a portion of a subject's body. A device includes a base with an LED therein, configured for emission of light therefrom for the light therapy at the same time as application of the cosmetic. The device includes a cover with a waveguide which, when aligned with the LED, is configured to pass light therethrough for the light therapy via emission of the light from the cover. The device can include circuitry configured for selection of a mode of operation for a corresponding light therapy.

### SUMMARY

In an aspect, the disclosure provides a device configured for a topical light therapy, the device comprising: a base comprising a light-emitting diode (LED) operably connected with a power source; and a cover comprising a waveguide with an entrance and an exit; wherein attachment of the cover to the base aligns the waveguide with the LED, such that at least a portion of a light beam of the LED is transmitted from the entrance of the waveguide to the exit of the waveguide for the topical light therapy.

In another aspect, the disclosure provides a device configured for a topical light therapy, the device comprising: a base, comprising: a LED operably connected with a power source, and a base adapter configured for reversible attachment of a cosmetic thereto; and a cover configured for reversible attachment to the base; wherein at least a first portion of a light beam of the LED is transmittable from the base for the topical light therapy.

In embodiments, the device comprises: a base adapter configured for reversible attachment of a cosmetic to the base; a communication interface, a recharge interface, or both; circuitry configured for selection of a mode of operation of the device among a plurality of modes of operation of the device; a waveguide in the cover, wherein the waveguide comprises an entrance and an exit for transmission of at least the first portion of the light beam of the LED therethrough; or any combination thereof.

In embodiments, the LED is of a plurality of LEDs and the waveguide is of a plurality of waveguides of the cover. In embodiments, at least a second portion of the light beam is scattered from the waveguide at a position of the waveguide between the entrance and exit. In embodiments, the second portion of the light beam creates an optical effect at least at the position of the waveguide between the entrance and exit when scattered therefrom.

In embodiments, the cosmetic is attached to the base, the device is configured for the topical light therapy in parallel with application of the cosmetic to a portion of a subject's body. In embodiments, the cosmetic is not attached to the base, the device is configured for the topical light therapy in absence of application of the cosmetic to a portion of a subject's body.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

FIG. 1A shows a perspective view of an example device configured for a topical light therapy, with a cover removed from a base, according to aspects of the disclosure.

FIG. 1B shows a perspective view of an example device configured for a topical light therapy, with a cover removed from a base and shown with transparent walls to reveal waveguides of the cover, as well as a cosmetic, according to aspects of the disclosure.

FIG. 1C shows a perspective view of an example device configured for a topical light therapy, with a cover removed from a base and shown with a cosmetic attached to the base, according to aspects of the disclosure.

FIG. 1D shows a plan view of a base of an example device with a cosmetic attached thereto, configured for a topical light therapy, according to aspects of the disclosure.

FIG. 1E shows a perspective view of an example device configured for a topical light therapy, with a cover attached to a base, according to aspects of the disclosure.

FIG. 1F shows a plan view of a cover of an example device, attached to a base and configured for a topical light therapy, according to aspects of the disclosure.

FIG. 2A shows a schematic of several electronic elements of an example device configured for a topical light therapy, according to aspects of the disclosure.

FIG. 2B shows an illustration of several features of an example computational device for use with a device configured for a topical light therapy, according to aspects of the disclosure.

FIG. 3 shows an isometric perspective view of an example device configured for a topical light therapy, including a lipstick cosmetic, according to aspects of the disclosure.

The foregoing aspects and many of the attendant advantages of this invention will become more readily appreciated as the same become better understood by reference to the following detailed description, when taken in conjunction with the accompanying drawings.

### DETAILED DESCRIPTION

Topical light therapy, or phototherapy, can utilize light-emitting diodes (LEDs) for delivery of light to a topical portion of a subject, such as the skin, for medical or cosmetic applications. These approaches can utilize LEDs with frequencies of 415 nm (blue), 633 nm (red), and 830 nm (infrared), and can demonstrate effectiveness for treatment of acne vulgaris, wound healing, psoriasis, squamous cell carcinoma in situ, basal cell carcinoma, actinic keratosis, and in at least some instances, cosmetic applications.

Phototherapy devices for cosmetic purposes are generally configured for phototherapy only, and do not include additional functionalities. As a result, individuals using these devices need to utilize separate articles and devices for additional cosmetic applications, such as application of lipstick, application of makeup, and the like. The reliance on separate cosmetic devices for separate cosmetic functionalities results in these individuals having to store, transport, and operate a multitude of devices, which can be time-consuming, lead to disorganization and clutter, and can be costly.

Accordingly, there is a need for combination devices that implement phototherapeutic functionality in combination with cosmetic product functionality, including devices that are modular and that enable use of different cosmetic products in combination with a phototherapeutic aspect. The present disclosure addresses these and other long-felt and unmet needs in the art.

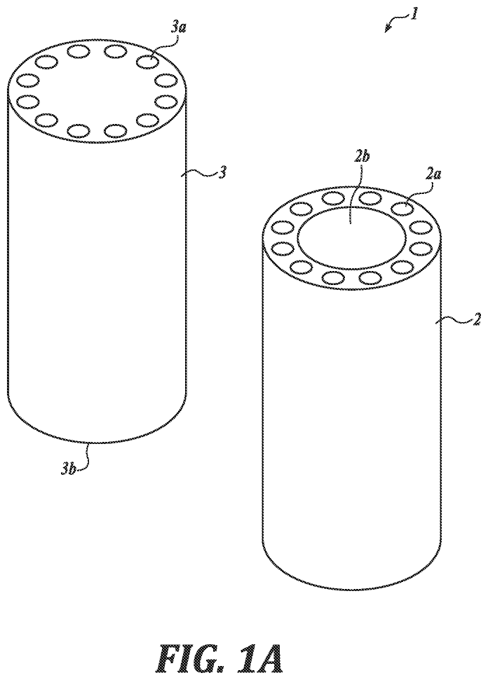

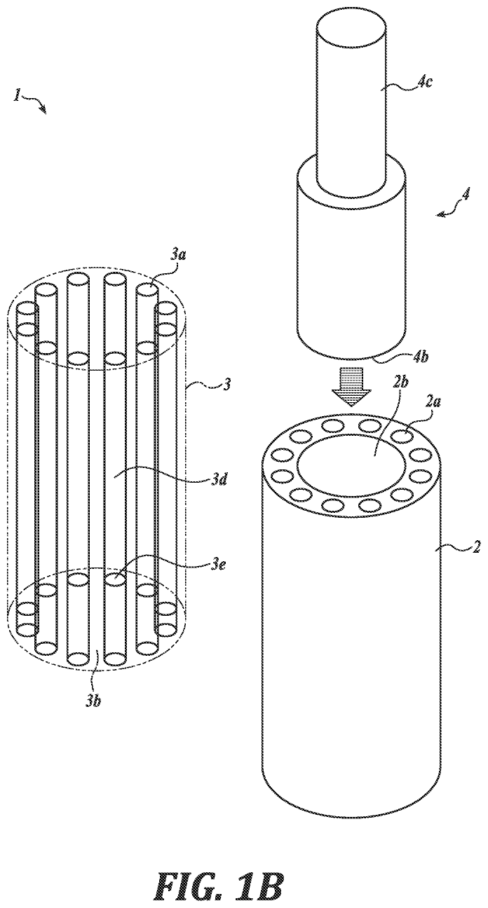

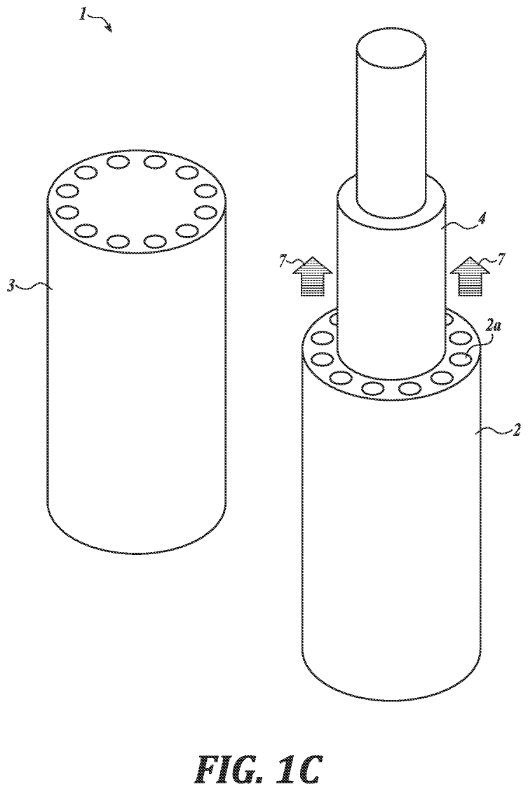

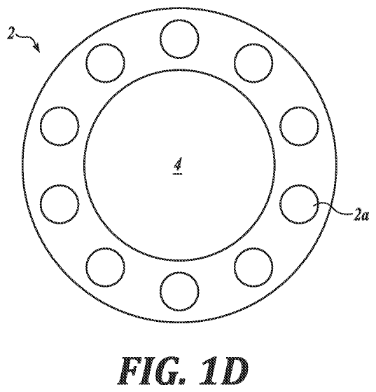

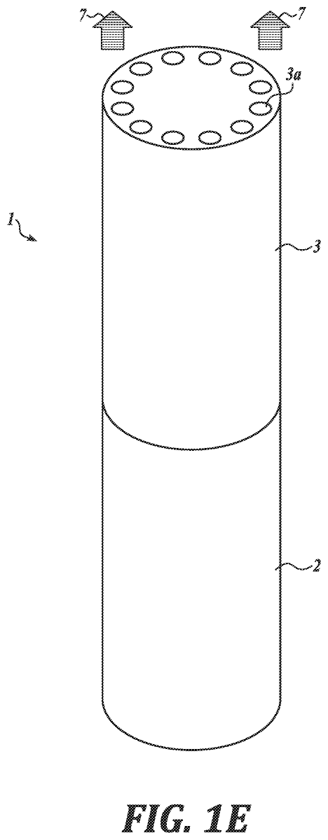

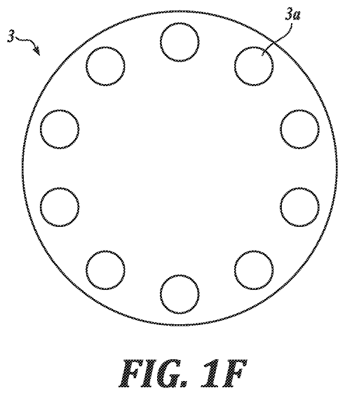

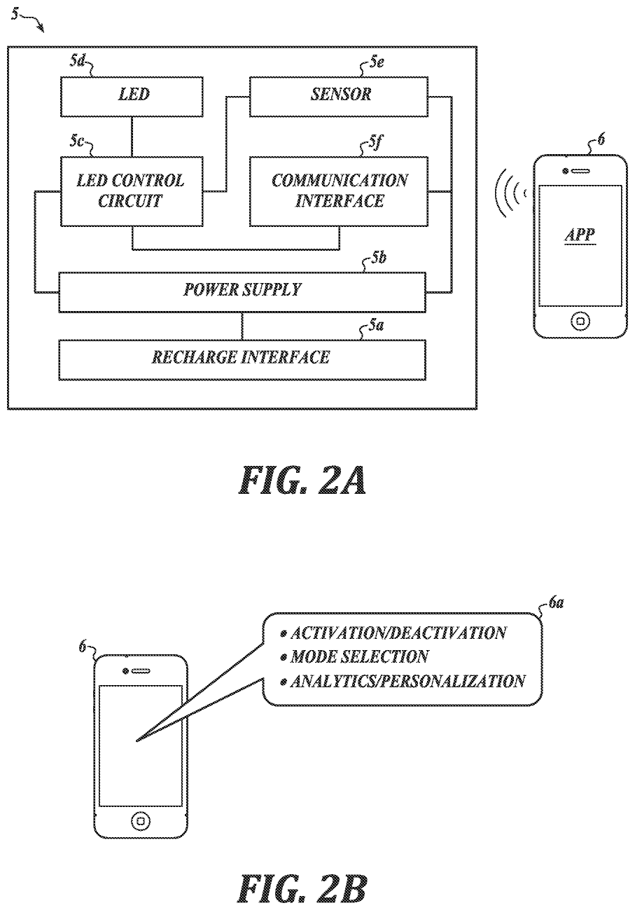

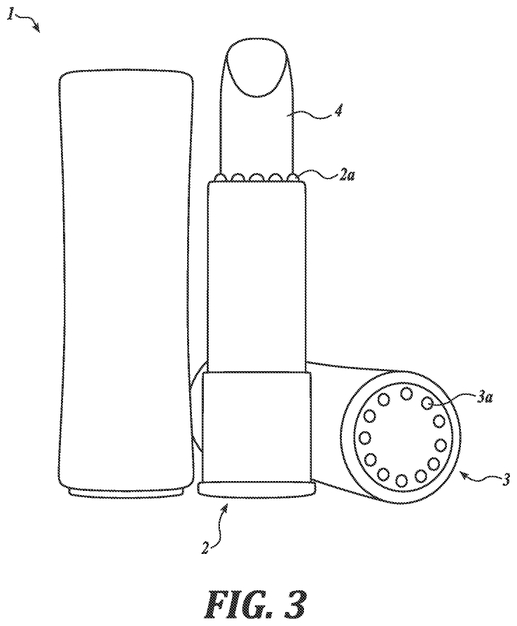

FIG. 1A shows a perspective view of an example device 1 configured for a topical light therapy, and FIG. 3 shows an isometric perspective view, with a cover 3 removed from a base 2, according to aspects of the disclosure. A device 1 is configured for a topical light therapy, and comprises: a base 2 comprising a light-emitting diode (LED) 2a operably connected with a power source (see, e.g., power supply 5b of FIG. 2A), and a cover 3 comprising a waveguide (see, e.g., 3d of FIG. 1B) with an entrance 3e and an exit 3a. Attachment of the cover to the base, as shown by way of example at FIG. 1E, aligns the waveguide with the LED, such that at least a portion of a light beam 7 of the LED is transmitted from the entrance 3e of the waveguide to the exit 3a of the waveguide, where the portion of the light beam 7 can contact a surface, e.g., a skin surface, for the topical light therapy.

In the shown embodiment, a cosmetic is not attached to the base 2, and the device 1 is configured for topical light therapy in the absence of application of the cosmetic to a portion of a subject's body. However, as further described herein, the device can also be configured for topical light therapy with the cosmetic attached to the base 2, as well as with attachment of the cover 3 to the base 2, providing a variety of configurations of the device 1 that are suitable for topical light therapy.

In embodiments, LEDs are positioned at or near a top of the base 2, such that light emitted from the LEDs is emitted directly to the environment adjacent to the LEDs when the LEDs are active. However, in embodiments, LEDs are positioned at or near a middle of the base 2, or alternatively, at or near a bottom of the base 2 (e.g., distal from base adaptor 2b). In such embodiments, waveguides can be positioned within the base 2 and connected to the LEDs, for example, such that light emitted from the LEDs is transported from the LEDs to the top of the base 2 for use in topical light therapy, and optionally, for further transmission through waveguides of the cover 3, as further described herein.

FIG. 1B shows the example device 1, with a cover 3 removed from a base 2 and shown with transparent walls (i.e., broken lines) to reveal waveguides 3d of the cover 3, as well as a cosmetic 4, according to aspects of the disclosure. Waveguides 3d can be positioned within the walls of the cover 3, such that there remains a volume within the cover for receipt of the cosmetic 4 therein. Base 2 includes LEDs 2a thereon, which emit light therefrom during operation. Base 2 also includes a base adapter 2b, configured for reversible attachment of the cosmetic 4 thereto by way of coupling with a cosmetic adapter 4b. The base adapter 2b and the cosmetic adapter 4b can include any mechanical interface that connects the cosmetic 4 to the base 2. Examples of such mechanical interfaces can include, but are not limited to, a snap-fit interface, a button interface, a clasp interface, a magnetic interface (e.g., in which the base adapter 2b and the cosmetic adapter 4b comprise magnetically complementary magnets), and the like. In the shown example, cosmetic 4 comprises a cosmetic consumable 4c thereon, which can include, for example, lipstick, lip balm, lip coloring, lip highlight, sunscreen, or another type of cosmetic consumable. With depletion of the cosmetic consumable 4c, it can be discarded and replaced with a new cosmetic consumable 4c for attachment to and use with the base 2, or can be refilled and reused, for example.

In the shown embodiment, there is a plurality of waveguides 3d within the cover 3. The waveguides 3d are optical waveguides, and are configured to transmit light emitted from the LEDs 2a therethrough, by way of entry through the waveguide entrances 3e and exit through the waveguide exits 3a. In at least some embodiments, the waveguides 3d can be comprised of one or more optical fiber waveguides, one or more transparent dielectric waveguides (e.g., made of plastic and glass), one or more liquid light guides, one or more liquid waveguides, or any combination thereof. In at least some embodiments, all or part of the cover 3 is at least partially optically transparent. In embodiments, at least a second portion of a light beam, emitted from the LEDs 2a, is scattered from one or more of the waveguides 3d at one or more positions of the waveguides between the entrance and exit, such that the scattered second portion of the light beam is transmitted out of the side of the cover 3. This can be used, for example, to introduce an optical or visual aesthetic.

As shown at FIG. 1C, the cover 3 can be removed from the base 2, and with the cosmetic 4 attached to the base 2, the device 1 can be used for topical light therapy in parallel with application of the cosmetic 4 to a portion of a subject's body. FIG. 1D shows a plan view of the base 2 with the cosmetic 4 attached thereto, configured for a topical light therapy. In embodiments, the LEDs can be activated for emission of the light beam 7 therefrom, and the light beam 7 can contact the subject's body, e.g., a portion of skin, during application of the cosmetic 4 to a portion of the subject's body. This can be effective, at least in part, due to the orientation and relative proximity of the LEDs to the portion of skin during application of the cosmetic. In this manner, a user can simultaneously apply the cosmetic 4 while also applying the topical light therapy with activation of the LEDs.

As shown at FIG. 1E, an alternate configuration of the device 1 for topical light therapy can include the cover 3 attached to the base 2 (e.g., either with or without a cosmetic position attached to the base 2 and within the cover 3). FIG. 1F shows a plan view of the device in this configuration. In this configuration, light emitted from the LEDs of the base 2 travels through the waveguides of the cover 3, and out the waveguide exits 3a. The portion of the light 7 that is emitted from the waveguide exits 3a can be used for topical light therapy, e.g., without concomitant application of cosmetic.

As shown at FIG. 2A, one or more electronic elements of an example device can be included, in a particular embodiment of the device. An example diagram 5 of such a device is shown, and includes a power supply 5b that is operably connected to a recharge interface 5a. Recharge interface 5a is configured to recharge the power supply 5b. Power supply 5b can include, for example, a rechargeable battery or capacitor. Recharge interface 5a can include, for example, a wired and/or wireless charging interface, and can be configured for transmission of power from an external power source to the power supply 5b to recharge the power supply 5b. Electrical power is delivered from the power supply 5b to other components of the device by way of one or more operable connections between the power supply 5b and various components.

While any battery or power supply 5b can be used for an example device, in at least some embodiments, a rechargeable battery is used and can be recharged with use of a charger operably connected with recharge interface 5a. Charging of the rechargeable battery by the charger can occur by a wired charging, for example, with physical electrical contacts between the rechargeable battery and the charger, for application of a voltage that originates from an external power source, such as an alternating current (AC) outlet, to recharge the rechargeable battery. However, in other implementations, charging of the rechargeable battery by the charger can occur by a wireless charging, for example, with a contactless or an electromagnetic coupling between the rechargeable battery and the charger. In such implementations, the charger can serve as a kind of charging station, at which the device can be placed near for inductive charging of the rechargeable battery by the charger. In these and other implementations, wireless charging can occur by way of passage of an alternating current through an induction coil in the charger, such that the moving electric charge creates a magnetic field that fluctuates in strength with fluctuation of amplitude of the electric current, and the changing magnetic field induces an alternating electric current in an induction coil of the device which passes through a rectifier for conversion to direct current (DC) which is used to recharge the rechargeable battery. While example wired and wireless battery recharging implementations are provided herein, any suitable wired or wireless charging implementation can be used without departing from the scope and spirit of the disclosure.

In the shown embodiment, a device can include an LED 5d, and optionally, an LED control circuit 5c. The LED 5d comprises one or more light-emitting diodes configured to emit light of any wavelength suitable for topical phototherapy, including but not limited to: blue light, yellow light, red light, near-infrared (near-IR) light, broad spectrum or white light, and any combination thereof. Blue light affects upper layers of skin, while yellow and red lights penetrate into deeper layers of the skin. Near-IR light penetrates even deeper into the skin. Combinations of light wavelengths emitted by the LED 5d can be used for phototherapy of combinations of depths within the skin.

Accordingly, as used herein, “topical” as used in “topical light therapy”, “topical phototherapy”, and the like, refers generally and inclusively to therapies that utilize light, including penetrative and non-penetrative light wavelengths for therapy at upper layers of skin as well as deeper layers of skin.

In embodiments, the LED control circuit 5c can be configured for control of the LED 5d according to a mode of operation of the device. Operation of the device can proceed according to a mode of operation, which can be a default mode or a mode selected by a user or medical professional, such as a dermatologist, for example. Parameters that can differ among modes of operation of the device can include, for example, wavelength (or band) of light emitted by an LED of the device, management of intensity of the wavelength of light emitted by the LED, management of time of emission of light by the LED, or any combination thereof. Activation of the wavelength of light can include activation of one or more LEDs that emit the desired wavelength, deactivation of one or more LEDs that do not emit the desired wavelength, or both; alternatively, activation of the wavelength of light can include use of one or more filters, such as one or more band-pass filters, to allow the desired wavelength (or band) to be transmitted therethrough for use. Management of intensity of the wavelength of light of the LED can include, for example, increasing voltage delivered to one or more LEDs that emit the desired wavelength (or band), decreasing voltage delivered to one or more LEDs that do not emit the desired wavelength, or both. Management of time of application of the light therapy can include a timer or timer circuit, for example, that activates one or more LEDs for a period of time, during which the phototherapy occurs. In embodiments, a mode of operation can be selected via operation of computational device 6.

In embodiments, the device includes a sensor 5e, configured for sensing a property or characteristic of the device or an operation of the device. For example, the sensor 5e can be configured to sense wavelength characteristics of light emitted from the LED 5d (or light emitted from the LED 5d and reflected back to the sensor 5e from a surface), an optical detection of a property or characteristic of a portion of a subject's skin, and the like.

In embodiments, the device includes a communication interface 5f, configured for electronic communication with computational device 6. The communication interface 5f can be configured for wired communication or wireless communication, for example, by way of a receiver or transceiver, for receipt of wireless signals from the computational device 6 and (in the case of a transceiver) for transmission of wireless signals to the computational device 6. Wireless signals received by the device from the computational device 6 can relate to operation of the device, for example, by activation of a mode of operation or manual activation of the device. Wireless signals sent by the device and received by the computational device 6 can relate to status or performance of the device, or can include sensor data obtained by the sensor 5e, for further analysis by the computational device 6 (or a networked or cloud computing device operably connected to the computational device 6, for example). Example wireless protocols that can be implemented by the communication interface 5f include but are not limited to: Bluetooth®, WiFi®, and the like. In these and other embodiments, appropriately configured computer hardware can be implemented with the communication interface 5f for enablement of the wireless protocols being implemented in the wireless connection with the computational device 6.

FIG. 2B shows an illustration of several features of an example computational device 6 configured for use with a device configured for a topical light therapy. In embodiments, a user can direct software of a computational device 6, which can include a smartphone or tablet or other personal computer device, for example, to control operation of the device according to various computational device functionalities 6a. For example, a user can activate and deactivate the device, select a mode of operation of the device, and view analytics or other personalization information related to operation of the device for topical phototherapy. In example embodiments during use, a user can configure a device for phototherapy, and use a functionality 6a of the computational device 6 to activate the device (e.g., activate one or more LEDs according to a mode of operation). The user can place the device near a portion of a subject's skin such that light emitted from the LEDs of the device contacts the subject's skin for phototherapy. At the conclusion of phototherapy, the user can use a functionality 6a of the computational device 6 to deactivate the device (e.g., deactivate one or more LEDs according to a mode of operation).

As such, in various aspects, the disclosure provides a “smart” cosmetic phototherapy device comprising circuitry configured to perform all or part of a method, including but not necessarily limited to control of LED operation. In embodiments, circuitry of a device is configurable with a processor and processor-executable instructions stored on a non-transitory machine-readable medium of the device. In embodiments, a device includes a software application configured to perform all or part of one or more methods or processes of the disclosure, in any order or combination. However, in embodiments, a device includes dedicated hardware circuitry. Further configuration of circuitry of the device can include wireless communication or networking circuitry, for example, circuitry configured for a wireless connection, such as a Bluetooth® connection, a Bluetooth® low energy (BLE) connection, and/or a Wi-Fi® connection, and/or a wired connection. The networking circuitry (e.g., communication interface 5f), in combination with other circuitry of the computational device, can be used to request, retrieve, and/or receive data from a computational device 6 or a remote server, for example. In embodiments, the device can be operated with use of a computational device 6, such as a smartphone or personal computational device, that can be operated by a user via a graphical user interface, as known in the art. In embodiments, the circuitry can include operable connection of one or more sensors with the processor, or other circuitry, for performing logic operations and/or methods based on data received from one or more sensors of the device.

### Tables

TABLE 1
Drawings references and descriptions.

| Reference | Description |
|---|---|
| 1 | Device |
| 2 | Base |
| 2a | Light-emitting diode (LED) |
| 2b | Base adapter |
| 3 | Cover |
| 3a | Waveguide exit |
| 3b | Cover adapter |
| 3d | Waveguide body |
| 3e | Waveguide entrance |
| 4 | Cosmetic |
| 4b | Cosmetic adapter |
| 4c | Cosmetic consumable |
| 5 | Device component diagram |
| 5a | Recharge interface |
| 5b | Power supply |
| 5c | LED control circuit |
| 5d | LED |
| 5e | Sensor |
| 5f | Communication interface |
| 6 | Computational device |
| 6a | Computational device functionality |
| 7 | Light |

### NON-LIMITING EMBODIMENTS

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. A device configured for a topical light therapy, the device comprising: a base comprising a light-emitting diode (LED) operably connected with a power source; and a cover comprising a waveguide with an entrance and an exit; wherein attachment of the cover to the base aligns the waveguide with the LED, such that at least a portion of a light beam of the LED is transmitted from the entrance of the waveguide to the exit of the waveguide for the topical light therapy.

Embodiment 2. The device of any other Embodiment, further comprising: a base adapter configured for reversible attachment of a cosmetic to the base.

Embodiment 3. The device of any other Embodiment, further comprising: a communication interface, a recharge interface, or both.

Embodiment 4. The device of any other Embodiment, further comprising: circuitry configured for selection of a mode of operation of the device among a plurality of modes of operation of the device.

Embodiment 5. The device of any other Embodiment, wherein the LED is of a plurality of LEDs and the waveguide is of a plurality of waveguides of the cover.

Embodiment 6. The device of any other Embodiment, wherein at least a second portion of the light beam is scattered from the waveguide at a position of the waveguide between the entrance and exit.

Embodiment 7. A device configured for a topical light therapy, the device comprising: a base, comprising: a LED operably connected with a power source, and a base adapter configured for reversible attachment of a cosmetic thereto; and a cover configured for reversible attachment to the base; wherein at least a first portion of a light beam of the LED is transmittable from the base for the topical light therapy.

Embodiment 8. The device of any other Embodiment, wherein the cosmetic is attached to the base, the device is configured for the topical light therapy in parallel with application of the cosmetic to a portion of a subject's body.

Embodiment 9. The device of any other Embodiment, wherein the cosmetic is not attached to the base, the device is configured for the topical light therapy in absence of application of the cosmetic to a portion of a subject's body.

Embodiment 10. The device of any other Embodiment, further comprising: a waveguide in the cover, wherein the waveguide comprises an entrance and an exit for transmission of at least the first portion of the light beam of the LED therethrough.

Embodiment 11. The device of any other Embodiment, wherein the LED is of a plurality of LEDs and the waveguide is of a plurality of waveguides of the cover.

Embodiment 12. The device of any other Embodiment further comprising: a communication interface, a recharge interface, or both.

Embodiment 13. The device of any other Embodiment, further comprising: circuitry configured for operable selection of a mode of operation of the device among a plurality of modes of operation of the device.

Embodiment 14. The device of any other Embodiment, wherein at least a second portion of the light beam is scattered from the waveguide at a position of the waveguide between the entrance and exit.

Embodiment 15. The device of any other Embodiment, wherein the second portion of the light beam creates an optical effect at least at the position of the waveguide between the entrance and exit when scattered therefrom.

While illustrative embodiments have been illustrated and described, it will be appreciated that various changes can be made therein without departing from the spirit and scope of the invention.

The embodiments of the invention in which an exclusive property or privilege is claimed are defined as follows:

1. A device configured for a topical light therapy, the device comprising:
a base comprising a light-emitting diode (LED) operably connected with a power source; and
a cover comprising a waveguide with an entrance and an exit;
wherein attachment of the cover to the base aligns the waveguide with the LED, such that at least a portion of a light beam of the LED is transmitted from the entrance of the waveguide to the exit of the waveguide for the topical light therapy.

2. The device of claim 1, further comprising: a base adapter configured for reversible attachment of a cosmetic to the base.

3. The device of claim 1, further comprising: a communication interface, a recharge interface, or both.

4. The device of claim 1, further comprising: circuitry configured for selection of a mode of operation of the device among a plurality of modes of operation of the device.

5. The device of claim 1, wherein the LED is of a plurality of LEDs and the waveguide is of a plurality of waveguides of the cover.

6. The device of claim 1, wherein at least a second portion of the light beam is scattered from the waveguide at a position of the waveguide between the entrance and exit.

7. A device configured for a topical light therapy, the device comprising:
a base, comprising: a LED operably connected with a power source, and a base adapter configured for reversible attachment of a cosmetic thereto; and
a cover configured for reversible attachment to the base;
wherein at least a first portion of a light beam of the LED is transmittable from the base for the topical light therapy.

8. The device of claim 7, wherein the cosmetic is attached to the base, the device is configured for the topical light therapy in parallel with application of the cosmetic to a portion of a subject's body.

9. The device of claim 7, wherein the cosmetic is not attached to the base, the device is configured for the topical light therapy in absence of application of the cosmetic to a portion of a subject's body.

10. The device of claim 7, further comprising: a waveguide in the cover, wherein the waveguide comprises an entrance and an exit for transmission of at least the first portion of the light beam of the LED therethrough.

11. The device of claim 10, wherein the LED is of a plurality of LEDs and the waveguide is of a plurality of waveguides of the cover.

12. The device of claim 7, further comprising: a communication interface, a recharge interface, or both.

13. The device of claim 7, further comprising: circuitry configured for operable selection of a mode of operation of the device among a plurality of modes of operation of the device.

14. The device of claim 10, wherein at least a second portion of the light beam is scattered from the waveguide at a position of the waveguide between the entrance and exit.

15. The device of claim 14, wherein the second portion of the light beam creates an optical effect at least at the position of the waveguide between the entrance and exit when scattered therefrom.
